(19) United States
(12) Patent Application Publication
(10) Pub. No.: US 2011/0112332 A1
(43) Pub. Date: May 12, 2011

(54) PROCESS FOR INCREASING THE COALESCENCE RATE FOR AMINE-INITIATED POLYETHERS

(75) Inventors: Sunil K. Chaudhary, Missouri City, TX (US); Jean P. Chauvel, Lake Jackson, TX (US); Christopher P. Christenson, Seguin, TX (US); Istvan Lengyel, Lake Jackson, TX (US); James P. Cosman, Sarnia (CA); John W. Weston, Sugar Land, TX (US); Katie Fischer, Rosharon, TX (US); David A. McCrery, Lake Jackson, TX (US)

(73) Assignee: Dow Global Technologies Inc., Midland, MI (US)

(21) Appl. No.: 13/001,628

(22) PCT Filed: Jul. 22, 2009
(86) PCT No.: PCT/US09/51404

§ 371 Date: Dec. 28, 2010

Related U.S. Application Data
(60) Provisional application No. 61/082,936, filed on Jul. 23, 2008.

(51) Int. Cl. C07C 41/34

(52) U.S. Cl. 568/623

(57) ABSTRACT

Disclosed is an improvement to a polyether preparation process that includes a coalescing step. Amine-initiated polyethers prepared using a mixed alkylene oxide feed tend to coalesce significantly more slowly than glycerin-initiated polyethers, particularly in processes that include a holding step and/or elevated temperature following an initial alkoxylation to form a pre-polymer. This improvement is to perform a remedial end-capping of the pre-polymer, which may include amine degradation products, using an alkylene oxide which contains at least (3) carbons, prior to the molecular weight-building alkoxylation with the mixed alkylene oxide feed. The rate and performance of coalescing thereafter may be substantially enhanced.

### BACKGROUND OF THE INVENTION

1. Technical Field

This invention relates to the production of polyethers, and in particular to a method for purifying a polyether to recover the polymerization catalyst therefrom.

2. Background of the Art

Polyethers are high volume chemical compounds that are used in a wide variety of applications including, for example, the preparation of polyurethanes and surfactants. A common method of making polyethers is to polymerize at least one alkylene oxide in the presence of an “initiator compound” and an alkali metal catalyst. Frequently, a low molecular weight pre-polymer of low viscosity is prepared first, and then used to manufacture the higher molecular weight polyether. In this way, polymers of the alkylene oxide may be prepared having a wide variety of molecular weights. The function of the initiator compound is to set the nominal functionality (number of hydroxyl groups per molecule) of the polyether.

In these processes it is often considered necessary in the industry to reduce the concentration of the alkali metal catalyst in the crude polyether to less than about 100 ppm. While a number of removal methods may be employed, one particularly convenient method includes adding water to the crude polyether, which initiates partitioning of the alkali metal catalyst into the water and results in formation of an emulsion. This emulsion is then allowed or enabled to continue separation into distinct phases via a step referred to as coalescing, and the polyether phase is isolated for final product recovery.

While a number of initiators are well known, among the most commonly employed are glycerin, sugars and amines While glycerin is useful in a number of standard commercial processes, amine initiator compounds have been shown to offer certain advantages in uses such as in preparing polyether compounds for polyurethane formulations. For example, U.S. Pat. No. 6,762,274 discloses a group of polyethers that are autocatalytic when used to form polyurethanes Amine-initiated polyethers are frequently employed in preparing flexible polyurethane foams, in particular, wherein they provide desirable properties such as consistency.

However, a particular problem has been encountered when amine-initiated polyethers are subjected to heterofeed (mixed feed) alkoxylations. Such alkoxylations generally include polymerizing the amine-initiated pre-polymer with a combination of different alkylene oxides, such as ethylene oxide, propylene oxide and/or butylene oxide, either concurrently or sequentially, thereby forming a random and/or block copolymer of a desired final molecular weight. In this case it has generally been found that the coalescence rate after the addition of water to extract the catalyst is substantially decreased. In fact, such rate may diminish to the point that coalescence and traditional separation methods are inadequate to achieve the desired product output. Since inefficient coalescence is associated with increased costs on a commercial scale, improvement of coalescence performance is widely sought by those skilled in the art.

### SUMMARY OF THE INVENTION

Accordingly, the invention provides, in one aspect, a process for preparing a polyether comprising alkoxylating, in the presence of an alkali metal catalyst, an amine initiator compound, having at least one active hydrogen-containing end-group, with at least one first alkylene oxide to form a pre-polymer; capping the pre-polymer by contacting it with at least one second alkylene oxide, having at least about 3 carbon atoms, to form a capped pre-polymer; alkoxylating the capped pre-polymer with a mixed feed of at least one third alkylene oxide and at least one fourth alkylene oxide to form a crude polyether; mixing the crude polyether with water to form an emulsion, the emulsion containing a dispersed aqueous phase containing the alkali metal catalyst, and a continuous polyether phase; coalescing the emulsion such that it forms a coalesced aqueous phase and a polyether phase; allowing or enabling the coalesced aqueous phase and the polyether phase to separate, such that the alkali metal catalyst is contained in the coalesced aqueous phase; and recovering the polyether phase as the final polyether; wherein the emulsion coalesces at a flux rate that is on average higher, or the amount of the alkali metal catalyst contained in the coalesced aqueous phase is lower, than in an otherwise-identical process in which the pre-polymer is not capped. This and other aspects are described more fully hereinbelow.

### DETAILED DESCRIPTION OF THE INVENTION

While the present invention may be used for preparing any polyether that is made from a pre-polymer, it is particularly useful to prepare polyethers that are amine-initiated and are subsequently heterofed alkoxylated. This is because this combination of processing parameters often results in formation, prior to the heterofeed alkoxylation, of at least one degradation product defined herein as an amine compound having at least one active hydrogen. The degradation product(s) tend to form when the pre-polymer is subjected to certain conditions, frequently of time, temperature, or both. Without wishing to be bound by any theory or hypothesis, it is suggested that these degradation products act as either surfactants themselves, or as precursors for surfactants, and that the resultant increase in the surfactancy of the crude polyether, in its various embodiments, operates to significantly diminish coalescence rate later on, following the final heterofeed alkoxylation.

The invention serves to reduce the negative effect of these degradation products on coalescence performance to a level that may be, in many non-limiting embodiments, comparable to that experienced for similarly-prepared, heterofed glycerin-initiated polyethers of comparable molecular weight. This reduces the overall production cost and cycle time, and therefore increases the commercial viability of the heterofed amine-initiated polyether product.

The invention provides, in one non-limiting embodiment, a polyether prepared by reacting an amine-containing initiator with at least one first alkylene oxide in the presence of an alkali metal polymerization catalyst. The preparation of polyethers via alkali metal-catalyzed polymerization of alkylene oxides is well known in the art and, except for the features described as critical herein, conventional alkylene oxide polymerization processes may be used to prepare a crude polyether final product hereunder.

The first alkylene oxide may be any that can be polymerized using an alkali metal polymerization catalyst, including, but not limited to, ethylene oxide, propylene oxide, 1,2-butylene oxide, 2,3-butylene oxide, 1,2-hexylene oxide, combinations thereof, and the like. Mixtures of two or more of the foregoing alkylene oxides may be used, and two or more of the foregoing alkylene oxides may be sequentially polymerized to form a block structure in the pre-polymer. Ethylene oxide, propylene oxide, 1,2-butylene oxide and 2,3-butylene oxide are generally preferred on the basis of cost, availability and properties of the resulting polyether. Use of mixtures of ethylene oxide and either propylene oxide or a butylene oxide isomer are also preferred, as is use of propylene oxide or a butylene oxide isomer followed by ethylene oxide, or of ethylene oxide followed by propylene oxide or a butylene oxide isomer, in sequential polymerization. Homopolymers of propylene oxide and polymers of mixtures of alkylene oxides containing propylene oxide are preferred polyethers, in particular and non-limiting embodiments.

The initiator compound contains one or more active hydrogen-containing groups. As used herein, an active hydrogen-containing group contains a hydrogen atom bonded to a heteroatom, and engages in a ring-opening reaction with an alkylene oxide. A carbon atom from the alkylene oxide becomes bonded to the heteroatom, and a hydroxyl group is formed. Among such active hydrogen-containing groups are carboxylic acid (—COOH), hydroxyl (—OH), primary amine (—NH₂), secondary amine (—NRH, where R is alkyl, especially lower alkyl), thiol (—SH), and the like, provided that at least one active hydrogen-containing group is a primary amine (—NH₂) or a secondary amine (—NRH, where R is alkyl, especially lower alkyl). The structure of the initiator compound is desirably selected to provide a desired functionality (i.e., number of hydroxyl groups per molecule) in the finished product and, in some cases, to provide desirable functional properties. For example, an initiator having a hydrophobic group may be selected if surfactant properties are desired in the product polyether. Among the many suitable initiator compounds are, for example, aliphatic and aromatic unsubstituted or N-mono-, N,N′-dialkyl and N,N′,N′-triialkyl-substituted diamines having 1 to 5 carbon atoms in the alkyl group, such as unsubstituted or mono- or dialkyl-substituted compounds such as ethylenediamine, diethylenetriamine, triethylenetetramine, tripropylenediamine, 1,3-propylenediamine, 1,3- and 1,4-butylenediamine, tetrapropylenepentamine, 1,2-, 1,3-, 1,4-, 1,5- and 1,6-hexamethylenediamine; N-(2-aminoethyl)-morpholine, N-(3-aminopropyl)-morpholine, N-(2-aminoethyl)-piperidine, N-(3-aminopropyl)-piperidine, N-(3-aminopropyl)-N′-n-propyl piperazine, and aminoethylpiperazine; aromatic mono- and polyamines such as toluenediamine, phenylenediamines, 1,3-, 1,4- and 2,6-tolylenediamine, 4,4′-, 2,4′- and 2,2′-diaminodiphenylmethane; alkanolamines such as ethanolamine, N-methyl- and N-ethyl-diethanolamine, and ammonia; combinations thereof; and the like. In one embodiment, the initiator may be at least one of the formula

HₘA—(CH₂)ₙ—N(R)—(CH₂)ₚ—AHₘ Formula I

wherein n and p are independently integers from 2 to 12; A at each occurrence is independently oxygen, nitrogen, sulphur or hydrogen, provided that only one of A may be hydrogen; R is a C₁ to C₃ alkyl group; m is zero when A is hydrogen, m is 1 when A is oxygen or sulphur, and m is 2 when A is nitrogen. The initiator may alternatively be at least one of the formula

H₂N—(CH₂)ₘ—N—(R)—H Formula II

wherein m is an integer from 2 to 12; and R is a C₁ to C₃ alkyl group. In additional embodiments suitable initiators may further include, for example, 3,3′-diamino-N-methyldipropylamine, 2,2′-diamino-N-methyldiethylamine, 2,3-diamino-N-methyl-ethyl-propylamine, N-methyl-1,2-ethane-diamine, N-methyl-1,3-propanediamine, N,N′-bis(3-aminopropyl)ethylenediamine and N-(3-aminopropyl)-N-methyl-propane-1,3-diamine; combinations thereof; and the like. Other examples of polyether polyols which are amine initiated and are useful in the present process may be found in, for example, U.S. Pat. Nos. 5,672,636; 5,482,979; and 5,476,969; and 6,762,274; which are incorporated herein by reference in their entireties.

The alkali metal polymerization catalyst is a compound that may displace a hydrogen atom from an active-hydrogen containing group on the initiator molecule. Suitable polymerization catalysts include alkali metal carbonates, alkali metal oxides, alkali metal hydroxides, and alkali metal salts of organic acids, such as potassium and sodium acetates, propionates, and the like. Preferred alkali metal polymerization catalysts are the alkali metal hydroxides, in particular potassium hydroxide, sodium hydroxide, barium hydroxide, cesium hydroxide, and combinations thereof. Cesium hydroxide is especially preferred in some non-limiting embodiments because it catalyzes the polymerization reaction under conditions that may reduce the degree of isomerization of propylene oxide to form monofunctional impurities.

Preparation of the final polyether of the invention begins by mixing at least one first alkylene oxide and the amine-containing initiator compound, under polymerization conditions and in the presence of the alkali metal catalyst, to form a pre-polymer. One method of adding the alkali metal catalyst is to mix a concentrated aqueous solution of the catalyst with some or all of the initiator compound. Such a concentrated aqueous solution advantageously contains from about 20 to about 60 weight percent, preferably from about 40 to about 55 weight percent, of the catalyst. Typically, from about 0.04 to about 0.2 moles of catalyst are used per equivalent of active hydrogen atoms in the initiator compound. In this way, a portion of the active hydrogen atoms in the initiator are reacted and replaced with alkali metal cations. Because the water tends to act as a difunctional initiator during the polymerization process, which is generally undesirable, it is customary to strip most or all of the water from the initiator/catalyst mixture prior to carrying out this first alkoxylation. However, the water may be left in the initiator if the presence of water-initiated polyether molecules in the final product is acceptable.

The polymerization is suitably conducted at an elevated temperature, for example, from about 80° C. to about 150° C. A pressure of from about 1 atmosphere (about 760 Torr) to about 10 atmospheres (about 7,600 Torr) is typically suitable. Generally the amount of the alkylene oxide may be from about 2 to about 4 moles, and, in certain non-limiting embodiments, about 3 moles, to about 1 mole of the active hydrogen-containing end-groups in the initiator compound. However, amounts ranging from about 1 mole to about 10 moles of total first alkylene oxide(s), per mole of active hydrogen-containing end-groups in the initiator compound, may be employed. It should be noted that it is desirable that the nature of the pre-polymer be such that the crude polyether to be eventually prepared therefrom be sufficiently insoluble in water that it may, in a subsequent step, form an emulsion with water that may then be separated into distinct polyether and aqueous phases via a coalescing step of some type.

The intermediate polyether, generally referred to herein as the pre-polymer, is prepared in anticipation of carrying out a further, main alkoxylation later. In the meanwhile, the pre-polymer is suitable for storing in a holding vessel for a period of time. Such is frequently done at an elevated temperature, to ensure that viscosity remains at a pumpable level. This temperature is frequently in excess of 80° C., and in some non-limiting embodiments in excess of 120° C. Storage is often continued for a time of from less than or equal to about 1 day to about 120 days, typically from about 15 days to about 45 days. Such storage may be necessitated by, for example, plant scheduling needs. While such storage and/or relatively high temperature may therefore be customary and/or necessary, undesirable side-effects may result. Such may include the formation of undesirable amine degradation products, as already discussed hereinabove.

Accordingly, an important benefit of the present invention is reduction of the effects of these degradation products on the coalescence rate, i.e., the invention serves to effectively speed up the coalescing part of the process, thereby shortening overall processing time. This benefit may be obtained by addition of a simple capping procedure, which may serve as simple, and economical, preventative insurance to ensure desirable output rate and/or a reduced level of alkali metal catalyst immediately following coalescence. The capping procedure may be employed, in particular non-limiting embodiments, after a holding period and/or subjection of the pre-polymer to elevated temperatures as discussed hereinabove. This capping procedure involves alkoxylation with preferably at least about 0.8 moles of propylene oxide, butylene oxide, or one or more other oxides with more than 3 carbon atoms, per mole of active hydrogen-containing end-groups in the pre-polymer, to form the capped pre-polymer. Such alkylene oxide(s) are termed herein the second alkylene oxide(s). In certain non-limiting embodiments, the capping involves use of from about 0.8 to about 10 moles of alkylene oxide(s) per mole of active hydrogen-containing end-groups in the pre-polymer. In certain other non-limiting embodiments, the capping involves use of from about 0.8 to about 5 moles of alkylene oxide(s) per mole of active hydrogen-containing end-groups in the prepolymer. This ratio range helps to ensure sufficient capping of the degradation product(s) as well as of the pre-polymer, without significant further polymerization at this point. Capping of the degradation products present in the pre-polymer appears to reduce the surfactancy of the products themselves, and/or their further formation of surfactant compounds. The result of this remedial step is a capped pre-polymer, which may alternatively be referred to as a capped intermediate to clarify the fact that, in some non-limiting embodiments, it includes both capped pre-polymer per se and any capped degradation product(s) therein, while in other non-limiting embodiments, there may be no significant amount of degradation product(s) present in the pre-polymer at the time of capping, and therefore no significant amount of capped degradation product(s) in the pre-polymer just prior to subjecting it to the main alkoxylation.

In some non-limiting embodiments it may be desirable to add additional alkali metal catalyst in order to facilitate the capping procedure. The relative reactivities of the materials should desirably be balanced against the fact that additional catalyst means that more catalyst ultimately must be removed from the crude polyether to form the final polyether, either during the coalescing step or in subsequent filterings.

Following the remedial capping step, the capped pre-polymer may then be subjected to its main alkoxylation, which in some non-limiting embodiments of the present invention may be a mixed, or heterofeed, alkoxylation. By “main alkoxylation” is meant the alkoxylation which ultimately brings the average molecular weight of the polyether to its desirable final level. This involves treating the crude polyether with at least two alkylene oxides, denominated a third alkylene oxide and a fourth alkylene oxide. These alkylene oxides may be fed concurrently or sequentially, in the presence of alkali metal polymerization catalyst, to result in a random or block copolymer polyether having an average molecular weight, in some non-limiting embodiments, from about 2,000 to about 5,000, and in other non-limiting embodiments, from about 800 to about 10,000. The main alkoxylation may be carried out under conditions and using equipment that is well known to those skilled in the art. In general, temperatures from about 80° C. to about 140° C., preferably from about 100° C. to about 130° C., may be used, and pressures may in many non-limiting embodiments be from atmospheric to superatmospheric. Again, as with the preparation of the pre-polymer and with the remedial capping step, higher pressures may be employed with higher temperatures in order to discourage the polymerization reaction mixture from boiling and, therefore, volatilizing and/or degrading at this point. Alkylene oxides selected as the third and fourth alkoxide may be any that are listed hereinabove as suitable for use as the first alkylene oxide, but are selected independently therefrom. The third and fourth alkoxides may not be identical to one another.

For this main alkoxylation, it is generally desirable for the alkylene oxides to be aggregately introduced in an amount of from about 3 to about 50 moles of alkylene oxide per moles of active hydrogen-containing end-groups on the initiator compound. In certain non-limiting embodiments, the alkylene oxides may be aggregately introduced in an amount of from about 10 to about 30 moles of alkylene oxide per mole of active hydrogen-containing end-groups on the initiator compound.

At the conclusion of the polymerization reaction, a crude polyether is obtained which contains residual alkali metal catalyst and, usually, a relatively small amount of unreacted alkylene oxide, in addition to the target polyether. The alkali metal catalyst exists at least partially in the form of alkoxide (—O⁻M⁺, where M represents the alkali metal) groups on the polyether.

In order to remove catalyst from the crude polyether according to the invention, the crude polyether may be mixed with sufficient water to extract the alkali metal catalyst. This is easily accomplished through agitation, the application of heat, or both. Agitation sufficient to finely disperse the water and polyether into each other may be accomplished using various types of mixing apparatus, such as, for example, stirred vessels, pin mixers, in-line agitators, impingement mixers, nozzle mixers, sonic mixers or static mixers. Elevated temperatures assist efficient extraction by reducing the solubility of water in polyether. Temperatures of from about 80° C. to about 150° C. are generally suitable for this purpose, with a temperature of from about 100° C. to about 140° C. being preferred. If a temperature above the boiling point of water is used, increased pressure is preferred in order to prevent boiling. Under these extraction conditions an emulsion of the water in the polyether is typically formed.

The amount of water that may be used in the extraction may vary widely. As little as about 3 percent, preferably at least about 5 percent, more preferably at least about 6 percent water, based on the weight of the crude polyether, may be employed. Up to about 100 percent or more of water may be used, based on the weight of crude polyether, but preferably no more than about 70 percent, more preferably no more than about 40 percent, and most preferably no more than about 20 percent of water. Using an unnecessarily large amount of water provides little or no benefit and requires the handling of larger volumes of materials.

In the extraction process, the alkoxide (—O⁻M⁺) groups generally react with water molecules to form hydroxyl groups and regenerate the corresponding alkali metal hydroxide, which migrates to, i.e., becomes dissolved in, the aqueous phase.

If the density of the water is close to that of the polyether, the water phase will separate slowly, if at all, from the polyether phase. Accordingly, a soluble inorganic salt or hydroxide may be added to the water in order to increase its density relative to that of the polyether phase. Suitable salts include soluble alkali metal salts, particularly potassium, sodium, or cesium salts. The alkali metal hydroxides are preferred, and it is often most convenient to use the same alkali metal catalyst that is used in forming the polyether. Among particularly useful alkali metal hydroxides are potassium hydroxide, sodium hydroxide, barium hydroxide, cesium hydroxide, and mixtures thereof, to increase the density of the water phase when needed. Sufficient salt or hydroxide may be added to create a density difference between the water and polyether phases of at least about 0.01 g/cc, more preferably at least about 0.02 g/cc. Up to about 10 percent, preferably up to about 5 percent, by weight of soluble salt or hydroxide, based on the weight of the water, is generally sufficient for this purpose.

Except for water and the optional addition of soluble salt or hydroxide, it is preferred not to include any other additives in the extraction portion of the process.

The emulsion generally formed in the extraction process may then be separated, or allowed to separate, using any means and/or method known to those skilled in the art. In one non-limiting embodiment, this may be accomplished via centrifugation. In another non-limiting embodiment, this may be accomplished by passing the emulsion through a coalescer medium. Either method may be suitable to effect coalescence of the finely dispersed droplets of water into larger agglomerations that, by virtue of their higher density relative to the polyether phase, will separate from the polyether to form a distinct water phase. Where centrifugation is employed, simple decantation may complete the separation. Where a coalescer medium is used, the product stream leaving the coalescer medium may contain enlarged water droplets in polyether, as compared to the mixture that is fed into the coalescer. The product stream may then be permitted to simply settle, whereupon the operation of gravity causes the agglomerated water and polyether droplets to separate into distinct water and polyether phases. This separation process may be promoted by holding the output from the coalescer bed under relatively quiescent conditions. Advantageously, a settling tank or an extension of the coalescer vessel is provided, to enable the product stream from the coalescer bed to be held under such relatively quiescent conditions until phase separation is complete. If desired, the emulsion may be contacted with two or more coalescer beds that are connected in series or in parallel, in order to obtain a more complete separation of the polyether and water phases.

The coalescer medium advantageously is in a form having a high surface area to volume ratio, such as a mesh, a fiber or a particulate. Particulate coalescing media are, in some non-limiting embodiments, preferred. When a particulate coalescer medium is used, the particle size is advantageously selected in conjunction with the density so that (1) the bed does not become fluidized, shift or develop uneven flow distribution; (2) a suitable pressure drop is developed across the coalescer bed; and (3) efficient coalescence is obtained. Those skilled in the art will be familiar with and/or easily able to determine appropriate configurations and constituencies of suitable coalescer beds. The diameter of the bed may be, in some non-limiting embodiments, advantageously selected for commercial applications to enable a flux across the surface in the range from about 800 lb/hr/ft² to about 3,000 lb/hr/ft².

In this manner, separate aqueous and polyether streams may be obtained. The aqueous stream contains at least about 90 percent by weight, preferably at least about 95 percent, more preferably at least about 98 percent, more preferably at least about 99 percent, and most preferably at least about 99.9 percent of the alkali metal polymerization catalyst contained in the crude polyether. The polyether phase will generally contain an amount of water (depending upon the solubility of the polyether in water) and also small amounts of organic by-products. This polyether phase is then recovered as the final polyether.

It is found, in certain non-limiting embodiments, that, when the process of the invention is compared with a process that omits the capping of the pre-polymer but is otherwise identical, the amount of the alkali metal polymerization catalyst, immediately post-coalescence, is reduced by at least about 25 percent. In other non-limiting embodiments, the reduction is at least about 50 percent. It is also found that the process of the invention may offer an increase in the average coalescence flux rate that is at least about 50 percent higher than that of a process that omits the capping of the pre-polymer but is otherwise identical. In other non-limiting embodiments, the flux rate for the inventive process is increased by at least about 100 percent, 200 percent, 300 percent, or even greater. Furthermore, because the remedial capping procedure can be accomplished quickly and inexpensively, while analytical testing to identify and quantify the presence of amine-containing degradation products is time-consuming and expensive, it may be expeditious in many commercial processes to institute use of the invention as a simple and relatively economical way to ensure acceptable coalescence performance.

Following coalescence, additional processing may be carried out to further reduce the concentration of the alkali metal catalyst, such as will be known or easily discernible to those of ordinary skill in the art. Such may include applications of heat and/or vacuum, filtration, and the like. Those skilled in the art will also be familiar with possible catalyst and water recycle options, according to the overall process.

The description hereinabove is intended to be general and is not intended to be inclusive of all possible embodiments of the invention. Similarly, the examples hereinbelow are provided to be illustrative only and are not intended to define or limit the invention in any way. Furthermore, those skilled in the art will be fully aware that other embodiments within the scope of the claims will be apparent, from consideration of the specification and/or practice of the invention as disclosed herein. Such other embodiments may include selections of specific initiators, alkylene oxides, catalysts, and combinations of such compounds; proportions of such compounds; mixing and reaction conditions, vessels, and protocols; performance and selectivity; additional applications of the products not specifically addressed herein; and the like; and those skilled in the art will recognize that such may be varied within the scope of the claims appended hereto.

### EXAMPLES

### Example 1

About 1 part of N-(3-aminopropyl)-N-methyl-propane-1,3-diamine, as an initiator, is transferred to a reactor vessel and then heated to about 140° C. About 1.17 part of propylene oxide is then added. This represents about 3 moles of propylene oxide per mole of the amine initiator, or about 80 grams per equivalent (g/eq). This is allowed to digest for about 15 minutes.

The temperature is then reduced to about 125° C., and about 0.27 part of a 46 percent aqueous solution of potassium hydroxide, KOH, is added. The water is quickly flashed off under vacuum to reach less than about 0.1 percent, resulting in a mixture now containing about 5.3 percent by weight of KOH. The temperature is then adjusted to about 120° C.

About 1.91 parts of propylene oxide is then fed into the mixture. This represents about 5 moles of propylene oxide per mole of the amine initiator, or about 150 g/eq. This is allowed to digest for about 15 minutes. At this time it is found that KOH concentration is about 2.9 percent by weight. This results in the pre-polymer, which is then transferred to a dedicated storage tank.

After a holding period of from about 15 to 60 days at a temperature of about 110° C., the pre-polymer is transferred to a reactor vessel and heated to about 110° C. Analysis at this point shows that a variety of degradation products are present including but not limited to C₃H₅—(PO)ₓ(EO)_{y}, wherein x is 2-10 and y is 0-5. About 3.25 parts of propylene oxide, representing about 2 moles of propylene oxide per mole of active hydrogen-containing end-groups in the pre-polymer, are fed in for about 40 minutes and then allowed to digest for about 60 minutes at 110° C. The result is the capped pre-polymer.

Then, about 21.26 parts of a heterofeed mixture of propylene oxide and ethylene oxide (about 17.95 parts PO, 3.31 parts EO), or about 1,000 g/eq, is fed in. This is allowed to digest at 110° C. for about 4.5 hours, to form the crude polyether.

To “finish” the polyether, the crude polyether is pumped out to a rundown tank while adding about 1.5 percent by weight water. More water is added to the batch, forming an emulsion while extracting KOH into the water phase. The emulsion is moved to a zirconium dioxide bed that acts as a coalescer unit. The denser water phase is separated by gravity and diverted to a recycle tank. Coalescer flux rate varies, on average, from about 1,500 to about 3,000 lbs/hr/ft², and the potassium hydroxide concentration in the crude polyether is less than about 50 ppm.

### Comparative Example 1

About 1 part of N-(3-aminopropyl)-N-methyl-propane-1,3-diamine, as an initiator, is transferred to a reactor vessel and then heated to about 140° C. About 1.17 part of propylene oxide is then added. This represents about 3 moles of propylene oxide per mole of the amine initiator, or about 80 grams per equivalent (g/eq). This is allowed to digest for about 15 minutes.

The temperature is then reduced to about 125° C., and about 0.27 part of a 46 percent aqueous solution of potassium hydroxide, KOH, is added. The water is quickly flashed off under vacuum to reach less than about 0.1 percent, resulting in a mixture now containing about 5.3 percent by weight of KOH. The temperature is then adjusted to about 120° C.

About 1.91 parts of propylene oxide is then fed into the mixture. This represents about 5 moles of propylene oxide per mole of the amine initiator, or about 150 g/eq. This is allowed to digest for about 15 minutes. At this time it is found that KOH concentration is about 2.9 percent by weight. This is the pre-polymer, which is then transferred to a dedicated storage tank.

After a holding period of from about 15 to 60 days at a temperature of about 110° C., the pre-polymer is transferred to a reactor vessel and heated to about 110° C. Analysis at this point shows that a variety of degradation products are present including but not limited to C₃H₅—(PO)ₓ(EO)_{y}, wherein x is 2-10 and y is 0-5.

Then about 24.51 parts of a mixture of propylene oxide and ethylene oxide (about 21.20 parts PO, 3.31 parts EO), or about 1,000 g/eq, is fed in to the (non-capped) pre-polymer. This is allowed to digest at 110° C. for about 4.5 hours, to form the crude polyether.

To “finish” the polyether, the crude polyether is pumped out to a rundown tank while adding about 1.5 percent by weight water. More water is added to the batch, forming an emulsion while extracting KOH into the water phase. The emulsion is moved to a zirconium dioxide bed that acts as a coalescer unit. The denser water phase is separated by gravity and diverted to a recycle tank. Coalescer flux rate is, on average, about 1,000 lbs/hr/ft². Potassium hydroxide concentration in the crude polyether is greater than about 100 ppm.

What is claimed is:

1. A process for preparing a polyether comprising
alkoxylating, in the presence of an alkali metal catalyst, an amine initiator compound, having at least one active hydrogen-containing end-group, with at least one first alkylene oxide to form a pre-polymer;
capping the pre-polymer by contacting it with at least one second alkylene oxide, having at least about 3 carbon atoms, to form a capped pre-polymer;
alkoxylating the capped pre-polymer with a mixed feed of at least one third alkylene oxide and at least one fourth alkylene oxide to form a crude polyether;
mixing the crude polyether with water to form an emulsion, the emulsion containing a dispersed aqueous phase containing the alkali metal catalyst, and a continuous polyether phase;
coalescing the emulsion such that it forms a coalesced aqueous phase and a polyether phase;
allowing or enabling the coalesced aqueous phase and the polyether phase to separate, such that the alkali metal catalyst is contained in the coalesced aqueous phase; and
recovering the polyether phase as the final polyether;
wherein the emulsion coalesces at a flux rate that is on average higher, or the amount of the alkali metal catalyst contained in the coalesced aqueous phase is lower, than in an otherwise-identical process in which the pre-polymer is not capped.

2. The process of claim 1 wherein the pre-polymer contains at least one amine-containing thermal degradation product.

3. The process of claim 1 wherein the pre-polymer is allowed to stand for a time period from about 1 to about 120 days, or subjected to a temperature of at least about 80° C., or both, prior to capping.

4. The process of claim 1 wherein the amine initiator compound is selected from the group consisting of alkylene amines, alkylene di- and triamines, and aromatic mono- and polyamines.

5. The process of claim 4 wherein the alkylene di- and triamines are selected from the group consisting of ethylenediamine, diethylenetriamine, aminoethyl-piperazine, 3,3′-diamino-N-methyldipropylamine, 2,2′-diamino-N-methyldiethylamine, 2,3-diamino-N-methyl-ethyl-propylamine, N-methyl-1,2-ethane-diamine, N-methyl-1,3-propanediamine, N,N′-bis(3-aminopropyl)ethylenediamine, N-(3-aminopropyl)-N-methyl-propane-1,3-diamine, and combinations thereof; and the aromatic polyamine is toluenediamine.

6. The process of claim 4 wherein the amine initiator compound is
at least one of the formula
HₘA—(CH₂)ₙ—N(R)—(CH₂)ₚ—AHₘ
wherein n and p are independently integers from 2 to 12; A at each occurrence is independently oxygen, nitrogen, sulphur or hydrogen, provided that only one of A may be hydrogen; R is a C₁ to C₃ alkyl group; m is zero when A is hydrogen, m is 1 when A is oxygen or sulphur, and m is 2 when A is nitrogen; or
at least one of the formula
H₂N—(CH₂)ₘ—N—(R)—H
wherein m is an integer from 2 to 12;and R is a C₁ to C₃ alkyl group.

7. The process of claim 1 wherein the alkali metal catalyst is selected from the group consisting of alkali metal carbonates, alkali metal oxides, alkali metal hydroxides, alkali metal salts of organic acids, and combinations thereof.

8. The process of claim 7 wherein the alkali metal hydroxide is selected from the group consisting of potassium hydroxide, sodium hydroxide, barium hydroxide and cesium hydroxide, and combinations thereof; and the alkali metal salts of organic acids are selected from the group consisting of potassium acetate, potassium propionate, sodium acetate, sodium propionate, and combinations thereof.

9. The process of claim 1 wherein the at least one first alkylene oxide and the at least one third alkylene oxide and the at least one fourth alkylene oxide are selected from the group consisting of ethylene oxide, propylene oxide, 1,2-butylene oxide, 2,3-butylene oxide, 1,2-hexylene oxide, and combinations thereof, provided that the at least one third alkylene oxide and the at least one fourth alkylene oxide are different from one another.

10. The process of claim 1 wherein the at least one second alkylene oxide is selected from the group consisting of propylene oxide, 1,2-butylene oxide, 2,3-butylene oxide, 1,2-hexylene oxide, and combinations thereof.

11. The process of claim 1 wherein a ratio of from about 1 to about 10 moles of the at least one first alkylene oxide, per mole of active hydrogen-containing end-groups in the amine initiator compound, is used.

12. The process of claim 1 wherein a ratio of from about 0.8 to about 5 moles of the at least one second alkylene oxide, per mole of active hydrogen-containing end-groups in the pre-polymer, is used.

13. The process of claim 1 wherein a ratio of from about 3 to about 50 moles of the at least one third alkylene oxide and the at least one fourth alkylene oxide, combined, per mole of active hydrogen-containing end-groups in the capped pre-polymer, is used.

14. The process of claim 13 wherein a ratio of from about 10 to about 30 moles of the at least one third alkylene oxide and the at least one fourth alkylene oxide, combined, per mole of active hydrogen-containing end-groups in the capped pre-polymer, is used.

15. The process of claim 1 wherein additional alkali metal catalyst is added to facilitate the capping of the pre-polymer.

16. The process of claim 15 wherein the alkali metal catalyst is selected from the group consisting of alkali metal carbonates, alkali metal oxides, alkali metal hydroxides, alkali metal salts of organic acids, and combinations thereof.

17. The process of claim 16 wherein the alkali metal hydroxide is selected from the group consisting of potassium hydroxide, sodium hydroxide, barium hydroxide and cesium hydroxide, and combinations thereof, and the alkali metal salts of organic acids are selected from the group consisting of potassium acetate, potassium propionate, sodium acetate, sodium propionate, and combinations thereof.

18. The process of claim 1 wherein the alkali metal catalyst contained in the coalesced aqueous phase is lower by at least about 25 percent.

19. The process of claim 18 wherein the alkali metal catalyst contained in the coalesced aqueous phase is lower by at least about 50 percent.

20. The process of claim 1 wherein the coalescer flux rate is higher on average by at least about 50 percent.
